(19) United States
(12) Patent Application Publication
(10) Pub. No.: US 2024/0320196 A1
(43) Pub. Date: Sep. 26, 2024

(54) SYSTEM FOR CONSTRUCTING DATABASE FOR STRUCTURE AND PROPERTY OF MATERIAL BASED ON BIG DATA AND DRIVING METHOD THEREOF

(71) Applicant: Foundation of Soongsil University-Industry Cooperation, Seoul (KR)

(72) Inventors: Jiwoong Kim, Seoul (KR); Hyokyeong Kim, Seoul (KR); Sohyun Park, Seoul (KR)

(73) Assignee: Foundation of Soongsil University-Industry Cooperation, Seoul (KR)

(21) Appl. No.: 18/224,578

(22) Filed: Jul. 21, 2023

(30) Foreign Application Priority Data
Mar. 20, 2023 (KR) 10-2023-0035576

(51) Int. Cl. G06F 16/21

(52) U.S. Cl. CPC G06F 16/211

(57) ABSTRACT

A system for constructing a database for a structure and a property of a material based on big data includes an input module that receives chemical information about the material based on the big data, a controller that obtains information about the structure and the property of the material by modeling the chemical information about the material based on quantum mechanics and calculating elastic constants, a database constructing module that constructs a database for the information about the structure and the property, and an output module that outputs the information about the structure and the property in the database with respect to a model selected through the modeling.

### CROSS-REFERENCE TO RELATED APPLICATIONS

A claim for priority under 35 U.S.C. § 119 is made to Korean Patent Application No. 10-2023-0035576 filed on Mar. 20, 2023, in the Korean Intellectual Property Office, the entire contents of which are hereby incorporated by reference.

### BACKGROUND

Embodiments of the inventive concept described herein relate to a system for constructing a database for a structure and a property of a material based on big data, and a driving method thereof.

In the past, properties of various materials, including mechanical, thermal, and electrical properties, have been obtained through experimental methods.

However, the above-described method not only consumes a lot of time and cost, but also has limitations because it is impossible to theoretically approach or identify the cause of material properties such as wear or high-temperature characteristics.

For this reason, only the development dependent on an empirical method is in progress, and the reality is that the development is focused on simple compounds or binary compositions.

In the meantime, although a calculation database for property information of the above-mentioned material is already present, it consists of only the material structure or energy information that is capable of being obtained by simple calculation.

In other words, it is difficult to obtain a large amount of digital property data due to the complexity of a model calculation method.

The inventive concept is derived from research conducted as part of Project for Research and Development with Middle Markets Enterprises and DNA (Data, Network, AI) Universities (Project No.; 1415182096, Project No.; P0021347, Research project name; Comprehensive material DB set-up and practical demonstration for the development of PVD thin film for machining high hardness and difficult-to-cut materials; Korea Institute for Advancement of Technology, task performing institution; Soongsil University, research period; 2022.04.01.˜2022.12.31.). Meanwhile, there is no property interest of the Korean government in any aspect of the inventive concept.

### SUMMARY

Embodiments of the inventive concept provide automation of a process of obtaining property information including structural properties, mechanical properties, and electrical properties of a material based on a quantum mechanical calculation method, and a process of constructing a database.

According to an exemplary embodiment, a system for constructing a database for a structure and a property of a material based on big data includes an input module that receives chemical information about the material based on the big data, a controller that obtains information about the structure and the property of the material by modeling the chemical information about the material based on quantum mechanics and calculating elastic constants, a database constructing module that constructs a database for the information about the structure and the property, and an output module that outputs the information about the structure and the property in the database with respect to a model selected through the modeling.

Moreover, according to an embodiment of the inventive concept, the chemical information includes a crystal structure and a type of a chemical substance constituting the material and an amount of the material.

Moreover, according to an embodiment of the inventive concept, the controller includes a modeling module that calculates the chemical information by using Ab initio calculation based on density functional theory, models the chemical information, and generates a quantum mechanical model, and a structure optimization module that calculates at least one free energy for the quantum mechanical model based on a density functional theory and extracts one quantum mechanical model having the lowest value among the at least one free energy.

Furthermore, according to an embodiment of the inventive concept, the controller further includes an elastic constant calculation module that generates at least one strained quantum mechanical model by performing modeling strain on the one quantum mechanical model, calculates the elastic constants and compliance for the one quantum mechanical model and the at least one strained quantum mechanical model, and obtains information about a property, and a noise removal module that removes noise by comparing the information about the property with experimental information about the property, which is previously entered.

Also, according to an embodiment of the inventive concept, the elastic constant calculation module obtains the information about the property including at least one of a bulk modulus, a shear modulus, and Young's modulus by using the elastic constant and the compliance.

Besides, according to an embodiment of the inventive concept, the controller further includes a property information extraction module that extracts only the information about the property from which noise is removed.

Moreover, according to an embodiment of the inventive concept, the property information extraction module calculates formation energy and cohesive energy for the one quantum mechanical model, and additionally obtains information about hardness and fracture toughness by using the elastic constant and the compliance.

Furthermore, according to an embodiment of the inventive concept, the database constructing module generates the database including at least one of a crystal structure, a type, the formation energy and the cohesive energy, a bulk modulus, a shear modulus, Young's modulus, the hardness and the fracture toughness of the one quantum mechanical model.

According to an exemplary embodiment, a driving method of a system for constructing a database for a structure and a property of a material based on big data includes receiving, by an input module, chemical information about the material based on the big data, obtaining, by a controller, information about the structure and the property of the material by modeling the chemical information about the material based on quantum mechanics and calculating elastic constants, constructing, by a database constructing module, a database for the information about the structure and the property, and outputting, by an output module, the information about the structure and the property from the chemical information based on the database.

Moreover, according to an embodiment of the inventive concept, a non-transitory computer-readable recording medium recorded with a program for executing the driving method of the system for constructing the database for the structure and the property of the material based on the big data.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a diagram of a system for constructing a database for a structure and property of a material based on big data, according to an embodiment of the inventive concept;

FIGS. 2A and 2B are diagrams related to modeling, according to an embodiment of the inventive concept;

FIGS. 3A and 3B are diagrams related to structure optimization of a quantum mechanical model, according to an embodiment of the inventive concept;

FIGS. 4A and 4B are diagrams related to modeling strain, elastic constants, and compliance, according to an embodiment of the inventive concept;

FIG. 5 is a diagram related to noise removal, according to an embodiment of the inventive concept;

FIG. 6 is a diagram of a database, according to an embodiment of the inventive concept; and

FIG. 7 is a flowchart of a driving method of a system for constructing a database for a structure and property of a material based on big data, according to an embodiment of the inventive concept.

### DETAILED DESCRIPTION

Hereinafter, various embodiments of the inventive concept will be described in detail with reference to the accompanying drawings such that those skilled in the art to which the inventive concept pertains may readily carry out the inventive concept. The inventive concept may be implemented in various different forms and is not limited to the embodiments described herein.

Components or elements not associated with the detailed description may be omitted to describe the inventive concept clearly, and the same reference numerals refer to the same or similar components throughout this application. Therefore, the reference numerals described above may be used in other drawings as well.

Moreover, the size and thickness of each of the components illustrated in the drawings are shown for convenience of explanation and the inventive concept is not necessarily limited thereto. In the drawing, the thickness may be exaggerated to clearly express various layers and regions.

Besides, an expression “the same” in the description may mean “substantially the same”. In other words, it may be the same to an extent that a person with ordinary knowledge may understand that it is the same. Other expressions may also be expressions in each of which “substantially” is omitted.

Furthermore, when a portion “comprises” a component in descriptions, it will be understood that it may further include another component, without excluding other components unless specifically stated otherwise. As used herein, a “˜unit” or “˜part” may be a unit that processes at least one function or operation and may refer to, for example, a software, FPGA, or hardware component. The function provided by the “˜unit” or “˜part” may be performed separately by a plurality of components, or it may be integrated with other additional components. The “˜unit” or “˜part” of this specification may not be necessarily limited to software or hardware, and may be configured to be included in an addressable storage medium, or may be configured to operate one or more processors. Hereinafter, embodiments of the inventive concept will be described in detail with reference to the drawings.

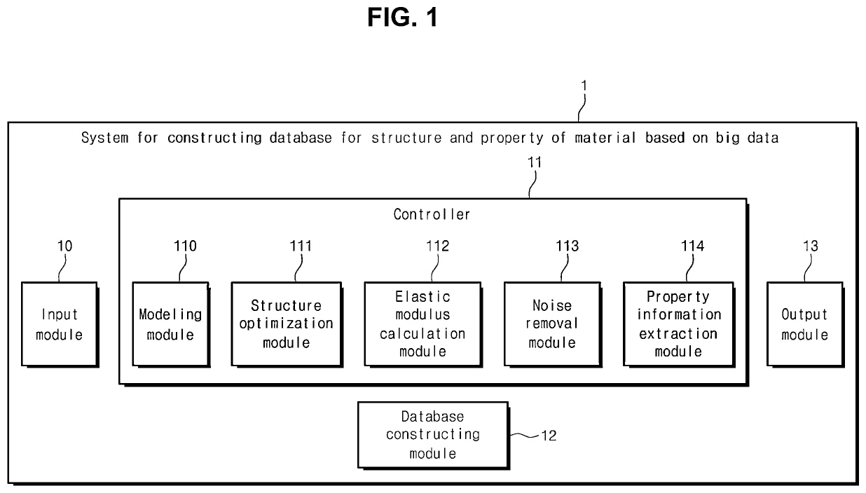

FIG. 1 is a diagram of a system for constructing a database for a structure and property of a material based on big data, according to an embodiment of the inventive concept.

According to an embodiment of the inventive concept, a system 1 for constructing a database for a property and structure of a material based on big data may include an input module 10, a controller 11, a database constructing module 12, and an output module 13.

However, to drive the system 1 for constructing a database for a property and structure of a material based on big data, the system 1 according to an embodiment of the inventive concept may include fewer or more components than the components illustrated in FIG. 1.

The input module 10 may receive chemical information about a material based on big data.

In this case, the chemical information may include the structure (or bond structure) and type of a molecule (or element) of a chemical substance constituting the material, and the amount (or addition amount) of the material.

The controller 11 may include a modeling module 110, a structure optimization module 111, an elastic modulus calculation module 112, a noise removal module 113, and a property information extraction module 114.

The controller 11 may model chemical information about a material based on quantum mechanics and may calculate elastic constants. The controller 11 may obtain information about the structure and property of the material by using elastic constants.

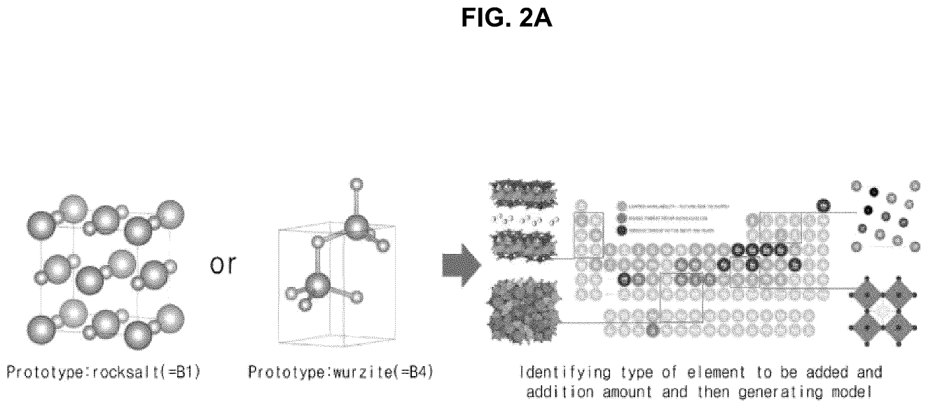

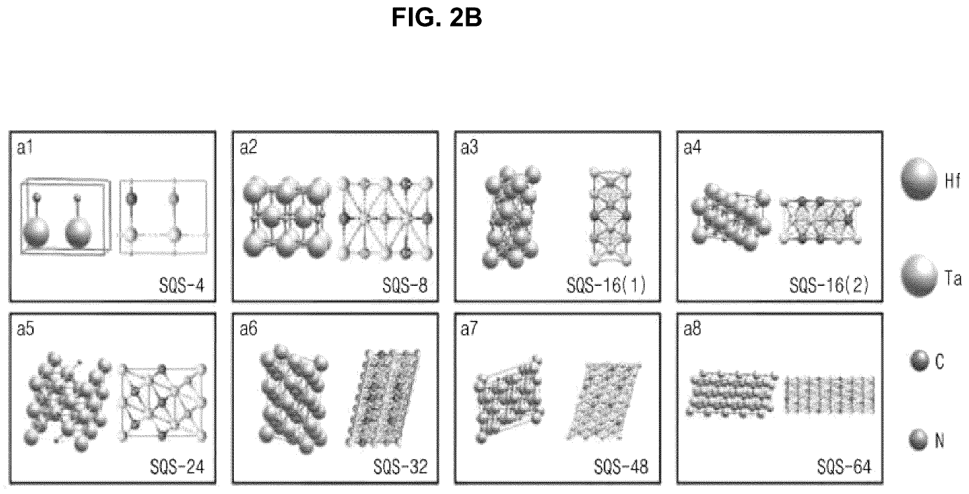

In detail, the modeling module 110 may generate a quantum mechanical model (see FIG. 2B) by calculating and modeling chemical information of a material in a quantum mechanical method.

The structure optimization module 111 may calculate at least one free energy for the quantum mechanical model based on density functional theory.

The structure optimization module 111 may extract one quantum mechanical model (stable equilibrium in FIG. 2B) having the lowest value among at least one free energy.

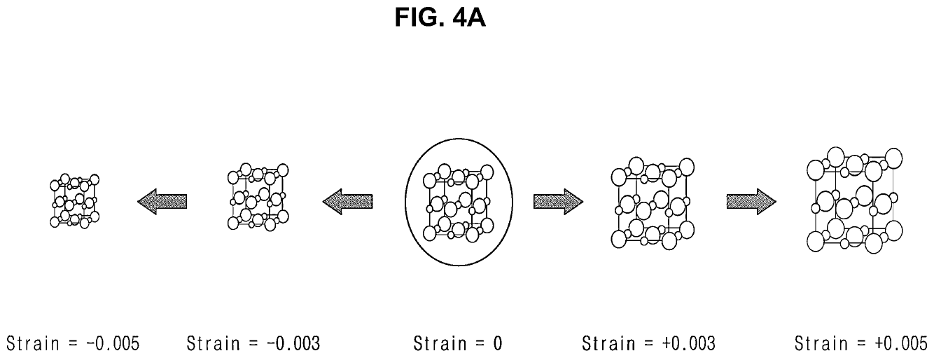

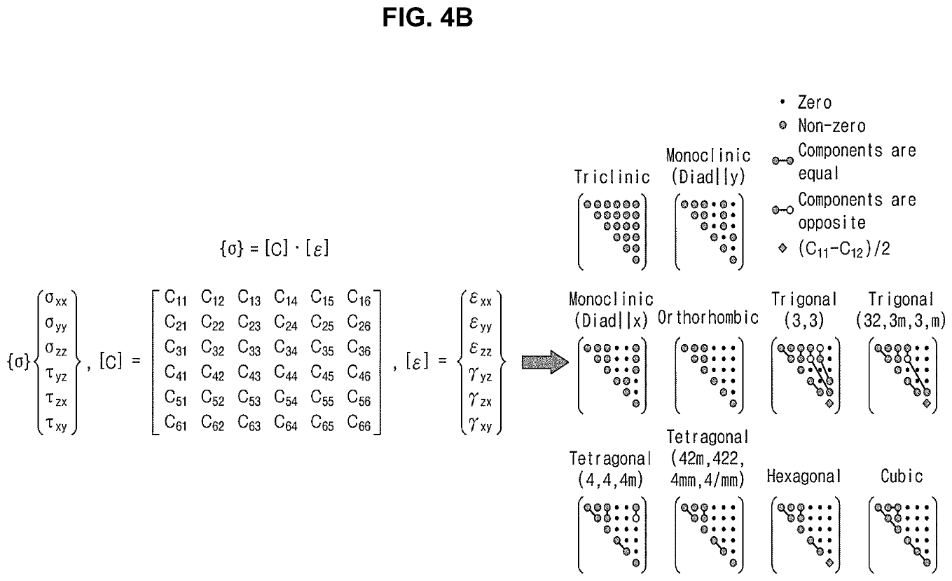

The elastic modulus calculation module 112 may generate at least one strained quantum mechanical model (see FIG. 4A) by performing modeling strain on one extracted quantum mechanical model.

The elastic modulus calculation module 112 may calculate elastic constants and compliance for one quantum mechanical model and at least one strained quantum mechanical model.

The elastic modulus calculation module 112 may obtain information about material properties by using the elastic constants and the compliance.

In detail, the elastic modulus calculation module 112 may obtain information about a property including at least one of bulk modulus, shear modulus, and Young's modulus by using the elastic constants and the compliance.

The noise removal module 113 may remove noise by comparing information about properties with experimental information about properties input in advance.

The property information extraction module 114 may extract only the information about properties from which noise is removed.

Moreover, the property information extraction module 114 may calculate formation energy and cohesive energy for one quantum mechanical model by using the elastic modulus and the compliance and may additionally obtain information about hardness and fracture toughness.

The database constructing module 12 may generate and construct a database including at least one of a structure (or bond structure) and type of a molecular (or atom), formation energy and cohesive energy, bulk modulus, shear modulus, Young's modulus, hardness and fracture toughness for any one quantum mechanical model.

The output module 13 may output the structure and properties from the chemical information input to the input module 10 based on the database.

FIGS. 2A and 2B are diagrams related to modeling, according to an embodiment of the inventive concept.

FIG. 2A is a diagram illustrating a process of modeling chemical information about a material based on quantum mechanics, according to an embodiment of the inventive concept. FIG. 2B is a diagram of a quantum mechanical model, according to an embodiment of the inventive concept.

Hereinafter, it will be described with reference to FIGS. 2A and 2B together.

The modeling module 110 may generate a probability-based model (or a quantum mechanical model), which is obtained by reflecting an irregular distribution of elements, by using chemical information about a material.

For example, an alloy theoretic automated toolkit (ATAT) program may be installed in advance in the modeling module 110.

The modeling module 110 may input the chemical information to the above-described ATAT and may select a prototype (Prototype (rocksalt (=B1) & wrutzite (=B4) crystal structure) of the quantum mechanical model.

Furthermore, the modeling module 110 may select the material to be a matrix and may generate the quantum mechanical model capable of performing ab initio quantum chemistry methods depending on the type of an element constituting the material, and an addition amount.

For example, the modeling module 110 may generate special quasi-random structures (SQS) for a material by using ATAT.

Alternatively, the modeling module 110 may model the material, which is in a state of disordered solid solutions, by using ATAT.

Alternatively, the modeling module 110 may model the material in a structure enumerating method by using ATAT.

Through the above-described process, the modeling module 110 may generate not only a simple structure model but also a high entropy material or solid solution model with a complex composition and may generate a model (or a quantum mechanical model) that randomly places and distributes elements.

Referring to FIG. 2B, the modeling module 110 may generating quantum mechanical models SQS-4, SQS-8, SQS-16(1), SQS-16(2), SQS-24, SQS-32, SQS-48, and SQS-64 according to the number of elements constituting the material by using ATAT, thereby improving accuracy.

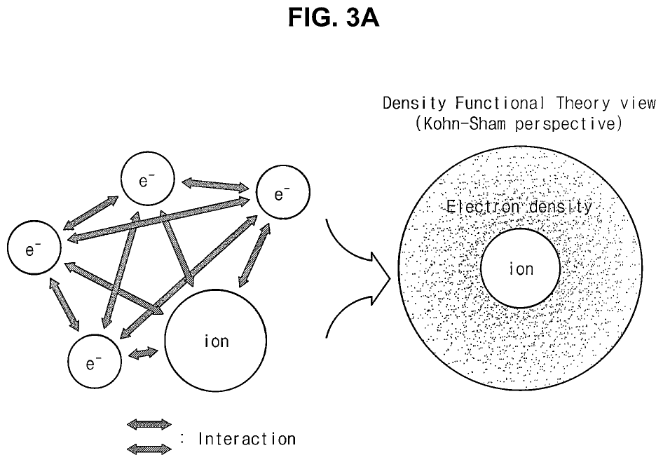

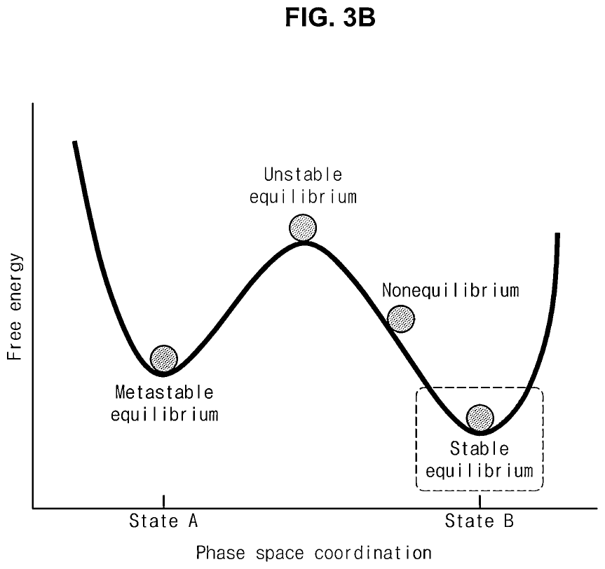

FIGS. 3A and 3B are diagrams related to structure optimization of a quantum mechanical model, according to an embodiment of the inventive concept.

FIG. 3A is a diagram of a process of calculating at least one free energy for a quantum mechanical model based on density functional theory, according to an embodiment of the inventive concept.

FIG. 3B is a diagram illustrating a process of selecting and optimizing a structure having the lowest value among at least one free energy, according to an embodiment of the inventive concept.

Hereinafter, it will be described with reference to FIGS. 3A and 3B together.

The structure optimization module 111 may calculate at least one free energy for the quantum mechanical model based on density functional theory.

In detail, the structure optimization module 111 may calculate interactions between all electrons constituting the quantum mechanical model based on the density functional theory. In this way, the structure optimization module 111 may calculate the electron density for the quantum mechanical model and may calculate at least one free energy based on the electron density.

At least one free energy calculated by the structure optimization module 111 may be shown in a graph in FIG. 3B.

The structure optimization module 111 may compare structures (metastable equilibrium, unstable equilibrium, nonequilibrium, and stable equilibrium) having at least one free energy with each other.

The structure optimization module 111 may extract one quantum mechanical model corresponding to a state (Stable B) in which free energy has the lowest value (stable equilibrium).

In other words, the structure optimization module 111 may extract one quantum mechanical model having a stable state with the lowest value (stable equilibrium) of free energy.

FIGS. 4A and 4B are diagrams related to modeling strain, elastic constants, and compliance, according to an embodiment of the inventive concept.

FIG. 4A is a diagram showing an example of generation of a strained quantum mechanical model (strain=−0.005, −0.003, +0.003, and +0.005) by performing modeling strain on one quantum mechanical model (strain=0), according to an embodiment of the inventive concept.

FIG. 4B is a diagram of a method for calculating elastic constants (C) and compliance depending on a crystal structure of the quantum mechanical model.

Hereinafter, it will be described with reference to FIGS. 4A and 4B together.

The elastic modulus calculation module 112 may generate at least one strained quantum mechanical model (strain=−0.005, −0.003, +0.003, and +0.005) by performing modeling strain on the one extracted quantum mechanical model, and may calculate elastic constants (C) and compliance for each model.

The elastic modulus calculation module 112 may calculate the elastic constants (C) and the compliance for one quantum mechanical model (strain=0) and at least one strained quantum mechanical model (strain=−0.005, −0.003, +0.003, and +0.005)

The elastic modulus calculation module 112 may determine and calculate the number of elastic constants (C) to be extracted for each crystal structure, through an energy calculation of one quantum mechanical model (strain=0) and at least one strained quantum mechanical model (strain=−0.005, −0.003, +0.003, or +0.005).

For example, referring to FIG. 4B, when the crystal structure is a cubic system, the elastic modulus calculation module 112 may calculate the elastic constants (C) of C11, C12, and C44.

Alternatively, when the crystal structure is a hexagonal system, the elastic modulus calculation module 112 may calculate the elastic constants (C) of C11, C12, C13, C33, C44, and C66.

In other words, required elastic constants may be different depending on the crystal structure.

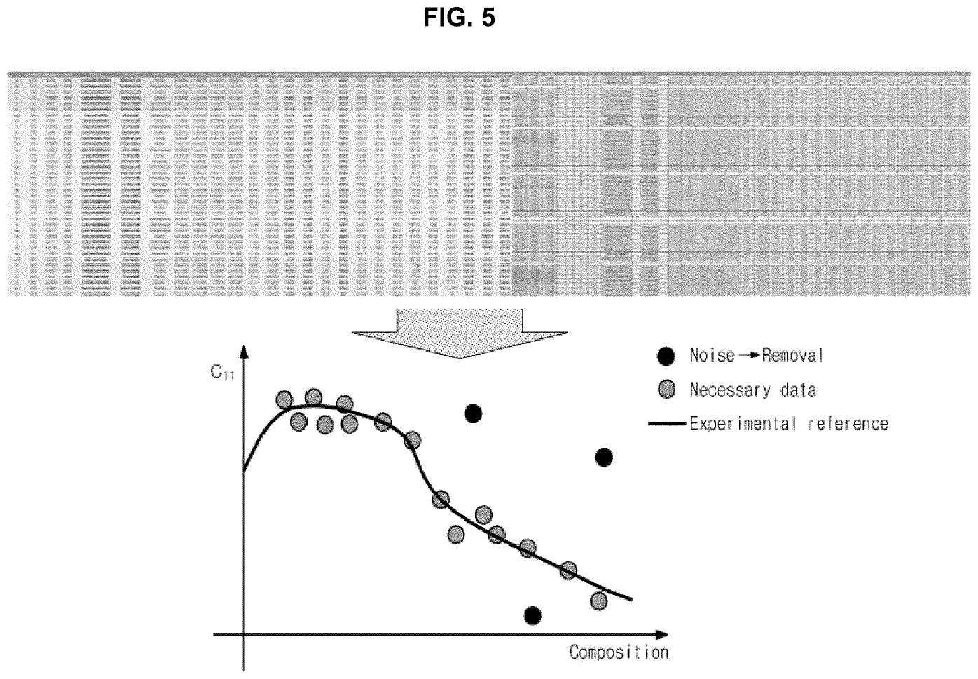

FIG. 5 is a diagram related to noise removal, according to an embodiment of the inventive concept.

The elastic modulus calculation module 112 may obtain information about a property including at least one of bulk modulus, shear modulus, and Young's modulus by using the elastic constants (C) and compliance, which are described in FIGS. 4A and 4B.

For example, bulk modulus (B) of a crystal structure being the cubic system may be expressed based on Equation 1 below.

$$Bulk modulus ( B ) = ( C 11 + 2 C 12 ) / 3 \qquad [ Equation 1 ]$$

Shear modulus (GV) may be expressed based on Equation 2 below.

$$Shear modulus ( GV ) = ( 3 C 44 + C 11 - C 12 ) / 5 \qquad [ Equation 2 ]$$

Shear modulus (GR) may be expressed based on Equation 3 below.

$$Shear modulus ( GR ) = 5 ( C 11 + C 12 ) C 44 / ( 4 C 44 + 3 ( C 11 - C 12 ) ) \qquad [ Equation 3 ]$$

Shear modulus (GH) may be expressed based on Equation 4 below.

$$Shear modulus ( GH ) = ( G V + GR ) / 2 \qquad [ Equation 4 ]$$

Young's modulus may be expressed based on Equation 5 below.

$$Young ' s modulus = 9 GB / ( 3 B + G ) \qquad [ Equation 5 ]$$

The noise removal module 113 may remove noise by comparing information about a property including at least one of the bulk modulus, the shear modulus, and the Young's modulus with experimental information (or experimental reference) about a previously input property.

The property information extraction module 114 may extract only the information (or necessary data) about a property from which noise is removed.

Moreover, the property information extraction module 114 may calculate formation energy and cohesive energy for one quantum mechanical model by using the elastic modulus and the compliance and may additionally obtain information about hardness and fracture toughness.

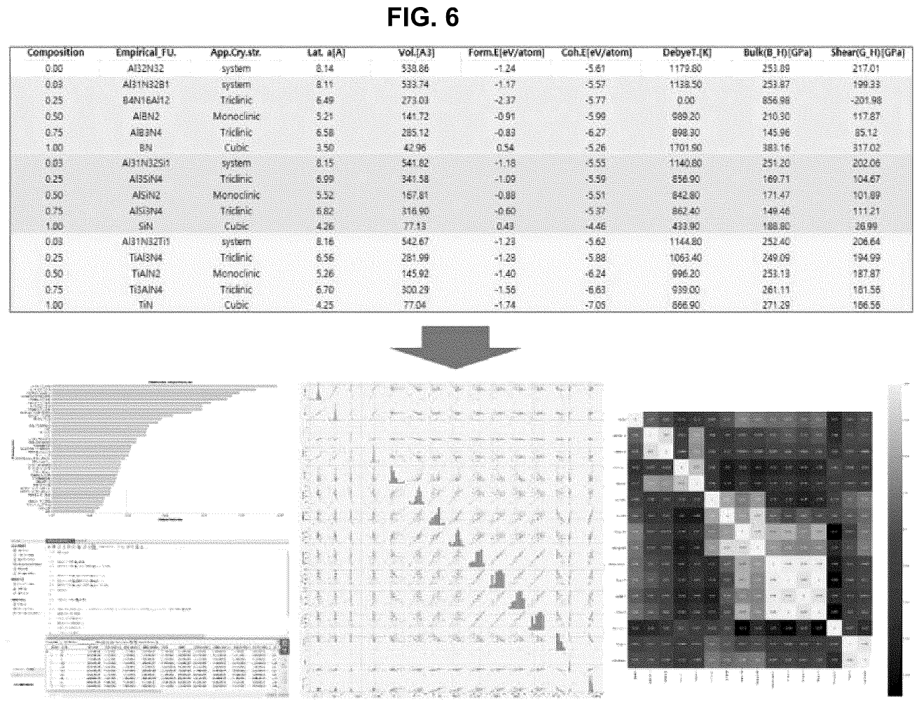

FIG. 6 is a diagram of a database, according to an embodiment of the inventive concept.

According to an embodiment of the inventive concept, the database constructing module 12 may generate and output a database including at least one of a structure (or bond structure) and type of a molecular (or atom), formation energy and cohesive energy, bulk modulus, shear modulus, Young's modulus, hardness and fracture toughness for any one quantum mechanical model, as a table of FIG. 6.

In other words, according to an embodiment of the inventive concept, the system 1 for constructing a database for a structure and a property of a material based on big data may construct the database including information about the structure and property of the material based on the big data in advance.

In this way, the output module 13 may output the structure and the property from chemical information about the material input from the input module 10 based on the database.

For example, when the chemical information about a material is entered into the input module 10 to develop a cutting tool for machining hard-to-cut materials, the output module 13 may output information about the structure and property of the material from the chemical information.

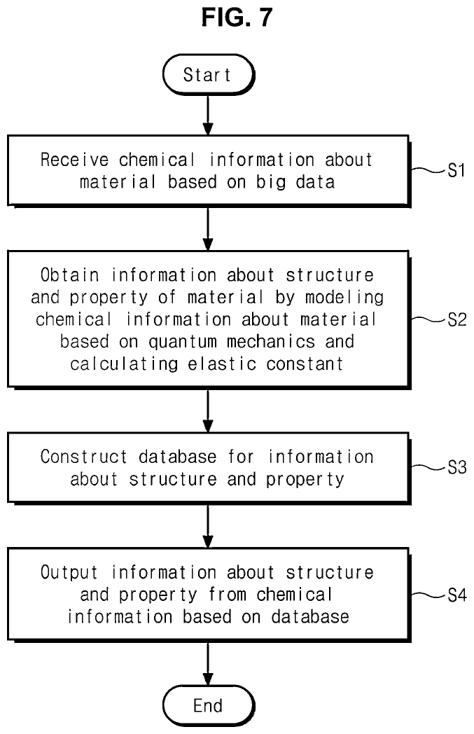

FIG. 7 is a flowchart of a driving method of a system for constructing a database for a structure and property of a material based on big data, according to an embodiment of the inventive concept.

In operation S1, the input module 10 may receive chemical information about a material based on big data.

In detail, the input module 10 may receive the chemical information including a structure (or bond structure) and type of a molecule (or element) of a chemical substance constituting the material, and the amount (or addition amount) of the material.

In operation S2, the controller 11 may obtain information about the structure and property of the material by modeling the chemical information about the material based on quantum mechanics and calculating elastic constants.

In detail, the controller 11 may model chemical information about the material based on quantum mechanics and may calculate the elastic constant. The controller 11 may obtain information about the structure and property of the material by using elastic constants.

In operation S3, the controller 11 may construct a database regarding the information about the structure and property.

In detail, the database constructing module 12 may generate and construct a database including at least one of a structure (or bond structure) and type of a molecular (or atom), formation energy and cohesive energy, bulk modulus, shear modulus, Young's modulus, hardness and fracture toughness for any one quantum mechanical model.

In operation S4, the output module 13 may output the information about the structure and property from the chemical information based on the database.

The drawings and detailed description of the invention given so far are merely illustrative of the inventive concept. This is only used for the purpose of describing the inventive concept and is not used to limit the scope of the inventive concept described in the meaning or claims. Therefore, it will be understood that various modifications and other equivalent embodiments are possible from this point by those skilled in the art. The technical protection scope of the inventive concept will be defined by the technical spirit of the appended claims.

The above-described embodiments may be implemented with hardware components, software components, and/or a combination of hardware components and software components. For example, the devices, methods, and elements described in the embodiments of the inventive concept may be implemented by using one or more general-use computers or special-purpose computers, such as a processor, a controller, an arithmetic logic unit (ALU), a digital signal processor, a microcomputer, a field programmable array (FPA), a programmable logic unit (PLU), a microprocessor, or any device which may execute instructions and respond.

A processing unit may perform an operating system (OS) or one or software applications running on the OS. Further, the processing unit may access, store, manipulate, process and generate data in response to execution of software. It will be understood by those skilled in the art that although a single processing unit may be illustrated for convenience of understanding, the processing unit may include a plurality of processing elements and/or a plurality of types of processing elements.

For example, the processing unit may include a plurality of processors or one processor and one controller. Also, the processing unit may have a different processing configuration, such as a parallel processor. Software may include computer programs, codes, instructions or one or more combinations thereof and configure a processing unit to operate in a desired manner or independently or collectively control the processing unit.

Software and/or data may be embodied in any type of machine, components, physical equipment, virtual equipment, computer storage media or devices so as to be interpreted by the processing unit or to provide instructions or data to the processing unit. Software may be dispersed throughout computer systems connected via networks and be stored or executed in a dispersion manner. Software and data may be recorded in one or more computer-readable storage media.

The methods according to the above-described embodiments may be recorded in a computer-readable medium including program instructions that are executable through various computer devices. The non-transitory computer-readable medium may include program instructions, data files, data structures, etc. independently or may include a combination thereof. The program instructions recorded in the media may be designed and configured specially for the exemplary embodiments of the inventive concept or be known and available to those skilled in computer software.

The computer-readable medium may include a hardware device, which is specially configured to store and execute program instructions, such as magnetic media (e.g., a hard disk drive, a floppy disk, and a magnetic tape), optical media (e.g., CD-ROM and DVD), read only memories (ROMs), random access memories (RAMs), and flash memories. Examples of computer programs include not only machine language codes created by a compiler, but also high-level language codes that are capable of being executed by a computer by using an interpreter or the like. The described hardware devices may be configured to act as one or more software modules to perform the operations of the above-described embodiments, or vice versa.

While embodiments have been shown and described with reference to the accompanying drawings, it will be apparent to those skilled in the art that various modifications and variations may be made from the foregoing descriptions. For example, adequate effects may be achieved even if the foregoing processes and methods are carried out in different order than described above, and/or the aforementioned elements, such as systems, structures, devices, or circuits, are combined or coupled in different forms and modes than as described above or be substituted or switched with other components or equivalents. Therefore, other implements, other embodiments, and equivalents to claims are within the scope of the following claims.

According to an embodiment of the inventive concept, a system for constructing a database for a structure and physical property of a material based on big data and a driving method thereof may automate not only a process of obtaining property information including structural features, mechanical features, and electrical features of a material based on a quantum mechanical calculation method, but also a process of constructing a database.

In this way, it is possible to minimize time and cost as well as theoretical approach to material properties and cause identification by breaking away from conventional experimental or empirical methods.

Moreover, property information may be obtained for complex compounds as well as simple compounds or binary compositions that are capable of being obtained by simple calculation regardless of the complexity of a calculation method, and large amounts of digital property data may be obtained.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A system for constructing a database for a structure and a property of a material based on big data, the system comprising:
an input module configured to receive chemical information about the material based on the big data;
a controller configured to obtain information about the structure and the property of the material by modeling the chemical information about the material based on quantum mechanics and calculating elastic constants;
a database constructing module configured to construct a database for the information about the structure and the property; and
an output module configured to output the information about the structure and the property in the database with respect to a model selected through the modeling.

2. The system of claim 1, wherein the chemical information includes a molecular structure and a type of a chemical substance constituting the material and an amount of the material.

3. The system of claim 1, wherein the controller includes:
a modeling module configured to calculate the chemical information by using Ab initio quantum chemistry methods based on the quantum mechanics in a quantum mechanical method, to model the chemical information, and to generate a quantum mechanical model; and
a structure optimization module configured to calculate at least one free energy for the quantum mechanical model based on a density functional theory and to extract one quantum mechanical model having the lowest value among the at least one free energy.

4. The system of claim 3, wherein the controller further includes:
an elastic constant calculation module configured to generate at least one strained quantum mechanical model by performing modeling strain on the one quantum mechanical model, to calculate the elastic constants and compliance for the one quantum mechanical model and the at least one strained quantum mechanical model, and to obtain information about a property; and
a noise removal module configured to remove noise by comparing the information about the property with experimental information about the property, which is previously entered.

5. The system of claim 4, wherein the elastic constant calculation module obtains the information about the property including at least one of a bulk modulus, a shear modulus, and Young's modulus by using the elastic constant and the compliance.

6. The system of claim 4, wherein the controller further includes:
a property information extraction module configured to extract only the information about the property from which noise is removed.

7. The system of claim 6, wherein the property information extraction module calculates formation energy and cohesive energy for the one quantum mechanical model by using the elastic constant and the compliance, and additionally obtains information about hardness and fracture toughness.

8. The system of claim 7, wherein the database constructing module generates the database including at least one of a molecular structure, a type, the formation energy and the cohesive energy, a bulk modulus, a shear modulus, Young's modulus, the hardness and the fracture toughness of the one quantum mechanical model.

9. A driving method of a system for constructing a database for a structure and a property of a material based on big data, the method comprising:
receiving, by an input module, chemical information about the material based on the big data;
obtaining, by a controller, information about the structure and the property of the material by modeling the chemical information about the material based on quantum mechanics and calculating elastic constants;
constructing, by a database constructing module, a database for the information about the structure and the property; and
outputting, by an output module, the information about the structure and the property from the chemical information based on the database.

10. A non-transitory computer-readable recording medium recorded with a program for executing the driving method of the system for constructing the database for the structure and the property of the material based on the big data in claim. 9.
